(19) United States
(12) Patent Application Publication
(10) Pub. No.: US 2007/0014864 A1
(43) Pub. Date: Jan. 18, 2007

(54) Novel pharmaceutical granulate

(75) Inventors: Ilan Zalit, Rosh-Ha-Ayin (IL); Julia Hrakovsky, Rosh-Ha-Ayin (IL); Ruth Tenengauzer, Hod Hasharon (IL); Sagit Shalom-Klein, Ramat Gan (IL)

Correspondence Address: KENYON & KENYON LLP, ONE BROADWAY, NEW YORK, NY, 10004, US

(73) Assignee: TEVA PHARMACEUTICAL INDUSTRIES, LTD.

(21) Appl. No.: 11/181,820

(22) Filed: Jul. 15, 2005

(51) Int. Cl. A61K 31/7012; A61K 31/55; A61K 31/415; A61K 31/401; A61K 31/366; A61K 31/365; A61K 31/22; A61K 31/495; A61K 9/14

(52) U.S. Cl. 424/489; 514/53; 514/253.06; 514/406; 514/217; 514/423; 514/460; 514/471; 514/548

(57) ABSTRACT

One of the objects of the invention relates to a pharmaceutical composition in the form of a granulate, wherein the granulates comprises an active pharmaceutical ingredient (API) having a poor water solubility intimately associated with at least one pharmaceutically acceptable sugar, and optionally or preferably at least one pharmaceutically acceptable excipient other than the at least one pharmaceutically acceptable sugar, wherein the active pharmaceutically ingredient has a water solubility less than about 20 mg/ml. The at least one pharmaceutically acceptable excipient other than the at least one pharmaceutically acceptable sugar is selected from the group consisting of disintegrants, wetting agents, diluents, binders, lubricants, glidants, coloring agents and flavoring agents. The at least one pharmaceutically acceptable sugar is preferably selected from pyranosyl pyranoses, such as lactose. Another object of the invention relates to a process for preparing a pharmaceutical granulate, comprising (a) combining an API having poor water solubility with a solution comprising at least one pharmaceutically acceptable sugar, for example a pyranosyl pyranose such as lactose, and a solvent, and optionally at least one pharmaceutically acceptable excipient other than the at least one pharmaceutically acceptable sugar to form a combined mixture; (b) drying the combined mixture of step (a); and (c) comminuting the product of step (b) to obtain the granulate.

### FIELD OF THE INVENTION

The present invention relates to granulates containing an active pharmaceutical ingredient having poor water solubility intimately associated with a pharmaceutically acceptable sugar, useful for pharmaceutical formulations, as exemplified by formulations of bicalutamide or fenofibrate suitable for tablets manufacture.

### BACKGROUND OF THE INVENTION

The solubility of an active pharmaceutical ingredient (API) influences the bioavailability of the drug and the dissolution of the drug can often set an upper limit on the rate of absorption of the drug. Many active pharmaceuticals have poor solubility in water and typically, thus lower bioavailability. Reduction in the particles size and concomitant increase in surface area of an active pharmaceutical ingredient has been used, with some success, to improve the dissolution of active pharmaceutical ingredients. However, this approach is limited by the particle size that can be achieved and by poor bulk flow and handling characteristics of finely powdered active pharmaceutical ingredients.

Strong milling of conventional granulates can increase the surface area of an active ingredient incorporated therein. This can result in a very powdery, difficult to handle fine powder. Re-granulation of this powder to improve handling can cause a reduction in surface area. Use of a higher content of a stiff binder can enable an increase in dissolution, presumably by increased surface area, but the extent of improvement is limited. In an anonymous article entitled “Formulations Comprising Lipid-Regulating Agents” published Jul. 11, 2002 and appeared in the August 2002 issue of IP.com Journal Volume 2 number 8, publication identifier ‘IPCOM000008767D’, there is mention made of “stronger” granules of fenofibrate made using “syrup solution of lactose” as a binder solution that with milling can increase the surface area and rate of dissolution, no significant detail is available that would enable one to achieve the results required and certainly no indication of the utility for any other API. The contents of this article are hereby incorporated in their entirety.

Clearly there is a need for improved methods for obtaining granulates of pharmaceutical compositions in which the active pharmaceutical ingredient exhibits the largest possible surface area to promote dissolution. The present inventors have surprisingly found that combining a solution of a sugar, e.g. lactose, with the ingredients of a pharmaceutical granulate formulation as herein described results in a granulate that can be comminuted to give a particulate that can be used to make an oral solid dosage form exhibiting surprisingly increased dissolution of the active pharmaceutical ingredient.

### SUMMARY OF THE INVENTION

One of the aspects of the invention concerns a granulate for a pharmaceutical composition, useful for making, among other things, oral solid dosage forms such as capsules and tablets, wherein the granulate comprises an active pharmaceutical ingredient (i.e. “API), which has poor water solubility, intimately associated with at least one pharmaceutically acceptable sugar, e.g., a pyranosyl pyranose such as lactose, and, optionally, at least one pharmaceutically acceptable excipient other than the at least one pharmaceutically acceptable sugar. The active pharmaceutical ingredient having poor water solubility includes fenofibrate, bicalutamide, atorvastatin, fluvastatin, simvastatin, candesartan, ezetimibe, oxcarbazepine, meloxicam, celecoxib, rofecoxib, valdecoxib, raloxifene, aripiprazole or glyburide. The at least one pharmaceutically acceptable excipient other than the at least one pharmaceutically acceptable sugar is preferably included in the granulate.

The present invention also relates to a process for making a granulate of a pharmaceutical composition, useful for making, among other things, oral solid dosage forms, comprising the steps of (a) combining an active pharmaceutical ingredient (i.e. “API”) having poor water solubility with a solution of at least one pharmaceutically acceptable sugar, for example a pyranosyl pyranose such as lactose, and optionally at least one pharmaceutically acceptable excipient other than the at least one pharmaceutically acceptable sugar to form a combined mixture, wherein the solution comprises the at least one pharmaceutically acceptable sugar and at least one solvent; (b) drying the combined mixture of step (a); and (c) comminuting the product of step (b) to obtain the granulate. The at least one pharmaceutically acceptable excipient other than the at least one pharmaceutically acceptable sugar is preferably included in the combining step (a). The at least one solvent in the solution of the at least one pharmaceutically acceptable sugar is preferably water.

Suitable pharmaceutically acceptable excipients include a polymer or copolymer of vinyl pyrrolidone and a wetting agent, for example sodium lauryl sulfate. Other pharmaceutically acceptable excipients, especially microcrystalline cellulose, can be and preferably are combined in the process. The product of comminution is termed a granulate and can be used directly as a pharmaceutical formulation, or it can be and preferably is blended with one or more additional pharmaceutically acceptable excipients prior to use, which can be one or more of pharmaceutically acceptable sugars and pharmaceutically acceptable excipients other than the one or more of pharmaceutically acceptable sugars. The advantages of the current invention are only realized when the granulate is the product of comminution as defined above

In another aspect, the present invention relates to a process for making a granulate of a pharmaceutical composition, useful for making, among other things, oral solid dosage forms whereby bicalutamide, a known non-steroidal anti-androgen agent,

is combined with a solution of at least one pharmaceutically acceptable sugar, for example lactose, and at least one pharmaceutically acceptable excipient other than the at least one pharmaceutically acceptable sugar. Suitable pharmaceutically acceptable excipients include a polymer or copolymer of vinyl pyrrolidone and a wetting agent, for example sodium lauryl sulfate. Other pharmaceutically acceptable excipients, especially microcrystalline cellulose, can be, and preferably are, combined in the process. The combined product is dried and comminuted to form a particulate. The particulate is a granulate of the present invention and can be used directly as a pharmaceutical formulation, or it can be and preferably is blended with one or more additional pharmaceutically acceptable excipients prior to use, which can be one or more of pharmaceutically acceptable dissaccahrides and pharmaceutically acceptable excipients other than sugars.

In yet another aspect, the present invention relates to a process for making a granulate of a pharmaceutical composition, useful for making, among other things, oral solid dosage forms, whereby an active pharmaceutical ingredient, especially bicalutamide, having poor water solubility is combined with microcrystalline cellulose, at least one non-crosslinked polymer of vinyl pyrrolidone, at least one disintegrant and wetting agent and an aqueous solution (for example, ca. 1:1, wt.:wt.) of lactose. The combined product is then dried and comminuted, for example by high-energy milling, to form a particulate that is a granulate of the present invention and can be used directly or it can be, and preferably is, blended with one or more additional pharmaceutically acceptable excipients prior to use, which can be one or more of pharmaceutically acceptable disaccharides and pharmaceutically acceptable excipients other than sugars.

In yet another aspect, the present invention relates to the granulate prepared by any of the processes described above.

In still another aspect, the present invention relates to a granulate prepared by combining bicalutamide, microcrystalline cellulose, croscarmellose sodium, povidone (polyvinylpyrrolidone, “PVP”), sodium lauryl sulfate and an aqueous solution of lactose monohydrate to form a combined product, drying the combined product, blending the dried combined product with colloidal silicon dioxide, and comminuting the resulting blend to obtain the granulate.

### BRIEF DESCRIPTION OF THE DRAWING

FIG. 1 depicts the dissolution profile of fenofibrate tablets prepared from Formulation 1 described below, and the dissolution profile of commercial fenofibrate tablets, Tricor 160 mg.

### DETAILED DESCRIPTION OF THE INVENTION

The present invention provides granulates of a pharmaceutical composition having an active pharmaceutical ingredient, i.e. a drug, having poor water solubility and a method for making the granulate. The granulates are useful for making oral solid dosage forms, for example capsules and compressed tablets in a variety of shapes. The advantages of the present inventive composition and method are notable with active pharmaceutical ingredients that have poor solubility in water. An API or drug is considered poorly water soluble if it has a solubility of less than about 20 mg per ml of water at about 25° C.

In this application, the term “active pharmaceutical ingredient, which has poor water solubility” or “active pharmaceutical ingredient having poor water solubility” means an API or drug having a solubility in water of less than about 20 mg per ml at about 25° C. Examples of the “active pharmaceutical ingredient, which has poor water solubility” or “active pharmaceutical ingredient having poor water solubility” include fenofibrate, bicalutamide, atorvastatin, fluvastatin, simvastatin, candesartan, ezetimibe, oxcarbazepine, meloxicam, celecoxib, rofecoxib, valdecoxib, raloxifene, aripiprazole or glyburide. Bicalutamide is a poorly water soluble active pharmaceutical agent particularly well suited for use in the present invention.

In the granulate for pharmaceutical composition of the invention, the active pharmaceutical ingredient (API) having poor water solubility and the at least one pharmaceutically acceptable sugar are intimately associated or in intimate association. The term “intimately associated” or “intimate association” refers to a state produced by a process comprising mixing the API and a solution of the at least one pharmaceutically acceptable sugar to form a mixture and drying the mixture. The API and the at least one pharmaceutically acceptable sugar in the dried mixture are intimately associated or in intimate association. The dried mixture in intimate association can be comminuted later to obtain granulates of an appropriate size. The state of being “intimately associated” or in “intimate association” is different from an ordinary state resulting from mixing powders of the API and powders of the at least one pharmaceutically acceptable sugar, optionally followed by compaction of the powder mixture. The intimate-association state differs from the ordinary state generated by mixing the API powders and sugar powders at least in that the at least one pharmaceutically acceptable sugar is more tightly adhered to the API in the intimate-association state than the ordinary state. In the intimate-association state of the pharmaceutical composition of the invention, the at least one pharmaceutically acceptable sugar forms a fairly or substantially continuous solid phase around a powder or granule of the API.

It is understood that pharmaceutical formulations comprising sugars not intimately associated with drugs were known in the pharmaceutical arts. In contrast, in the pharmaceutical composition of the invention, the API having poor water solubility and the at least one pharmaceutically acceptable sugar are in intimate association, with the API and the at least one pharmaceutically acceptable sugar combined in a matrix having a fairly or substantially continuous phase achieved by drying a mixture of the API and a solution of the at least one pharmaceutically acceptable sugar. The matrix of the intimate association of the API and the at least one pharmaceutically acceptable sugar achieves a consistency and stable adherence between the API and sugar(s) not achievable with the prior art process of mixing powders of the corresponding API with powders of the least one pharmaceutically acceptable sugar. As a result of the intimate association (and the milling of the dried granules), the pharmaceutical composition of the invention has superior dissolution properties than the prior art powder mix of the corresponding API and the at least one pharmaceutically acceptable sugar.

The pharmaceutical compositions of the invention are distinguished from the prior art products of classic lyophilization or freeze-drying where in contrast to the compositions of the present invention, the resultant product of that prior art technique generally results in a “cake” of a fluffy fragile matrix that can reportedly achieve improved dissolution by the “airy” and or porous nature of the matrix resulting from the lyophilization technique. It is however contemplated that, in the process for making the granulate of a pharmaceutical composition according to the present invention, the step of drying the combined mixture may include procedures where this drying is achieved at least partially by sublimation.

A poorly water soluble active pharmaceutical agent incorporated, by being intimately associated with at least one pharmaceutically acceptable sugar, into the granulate of the pharmaceutical composition of the present invention dissolves faster and to a greater extent in aqueous media than does the same poorly water soluble active pharmaceutical agent incorporated into a granulate or tablet made by conventional methods and/or by direct compression methods. The improved dissolution of the active pharmaceutical ingredient having poor water solubility in the granulate of the present invention, compared with the same active pharmaceutical ingredient incorporated into a granulate made by conventional methods, can be determined by tests conducted under conditions at least as stringent as using 1000 ml of a 1% aqueous solution of sodium lauryl sulfate at 37° C. with the USP paddle method rotating at 50 rpm and sampling time of 15, 30, 45 or 60 min, wherein if the active pharmaceutical ingredient is bicalutamide the amount of bicalutamide released is determined with a UV detector at 272 nm. For example, the granulate prepared by the method of the present invention can be fabricated into a compressed tablet and the dissolution of the active pharmaceutical ingredient determined by a suitable technique, for example dissolution test<711>of the United States Pharmacopoeia, and compared to the dissolution measured for a tablet compressed using conventionally produced granulate. When the active pharmaceutical ingredient is bicalutamide, a pharmaceutical dosage form comprising the granulate of the invention can have a dissolution property in which at least 50% of the bicalutamide dissolves in about 15 minutes, preferably at least about 65% of the bicalutamide dissolves in about 30 minutes, and more preferably at least 75% of the bicalutamide dissolves in about 45 minutes when tested under conditions at least as stringent as 1000 ml of a 1% aqueous solution of sodium lauryl sulfate at 37° C. using a USP paddle method rotating at 50 rpm when measured by a UV detector at 272 nm. For instance, when the active pharmaceutical ingredient is bicalutamide, the pharmaceutical dosage form of the invention can release about 80% of the bicalutamide in about 15 minutes or about 95% of the bicalutamide in about 30 minutes, when tested under conditions at least as stringent as 1000 ml of a 1% aqueous solution of sodium lauryl sulfate at 37° C. using a USP paddle method rotating at 50 rpm when measured by a UV detector at 272 nm.

In the instant patent application, the term “at least one pharmaceutically acceptable sugar” refers to a pharmaceutically acceptable monosaccharide, disaccharide or mixtures thereof, with the “at least one pharmaceutically acceptable sugar” comprises preferably at least a pharmaceutically acceptable disaccharide. Examples of the “at least one pharmaceutically acceptable sugar” include mannitol, sorbitol, glucose, fructose, galactose and, preferably, a disaccharide such as sucrose and, more preferably, a pyranosyl pyranose (e.g., maltose, isomaltose, cellobiose, melibiose, gentiobiose and, most preferably, lactose). The “at least one pharmaceutically acceptable sugar” to be combined with the API is in the form of a solution, preferably an aqueous solution or water/organic solution, in a sugar-to-solvent ratio generally between about 0.05:1 to about 1:0.05, preferably about 0.1:1 to about 1:0.1, more preferably about 0.5:1 to about 1:0.5 and most preferably about 1:1 (wt/wt; based on the total weight of the at least one pharmaceutically acceptable sugar: the weight of the solvent in the sugar solution). Depending on the API and the strength of the pharmaceutical dosage form comprising the granulate of the present invention, the weight ratio of the sugar (originated from the granulation solution) and the API having poor water solubility in the granulate can be from about 0.1:1 to about 1000:1, and preferably from about 0.1:1 to about 100:1 or from about 0.1:1 to about 10:1, e.g. about 0.5:1, about 1:1, about 2:1, about 3:1, about 5:1, about 10:1 or about 50:1. For relatively high dose products (e.g. pharmaceutical granules containing bicalutamide), the weight ratio of the sugar and the API having poor water solubility in the granule is preferably about 0.5:1 to about 5:1, e.g. about 0.5:1, about 1:1, about 2:1 or about 3:1, and more preferably about 0.7:1. In one of the embodiments, the present invention provides tablets comprising the granulate of the present invention, wherein the active pharmaceutical ingredient having poor water solubility in the granulate is bicalutamide, wherein the weight ratio of the sugar and bicalutamide can be about 0.7:1.

Many active pharmaceutical ingredients can be administered to a subject, particularly a human, in need of treatment with that active pharmaceutical ingredient in the form of an oral solid dosage form. Compressed tablets, in a variety of shapes, and filled capsules are examples of oral solid dosage forms. Oral solid dosage forms are rarely fabricated from neat active pharmaceutical ingredient. Rather, they are often fabricated of a granulate made by combining an active pharmaceutical ingredient with one or more pharmaceutically acceptable excipients. It is well known that pharmaceutically acceptable excipients can be broadly classified according to their intended function in the granulate or oral solid dosage form. One skilled in the art of pharmaceutical formulation knows that a given excipient may perform more than one function and the function of an excipient can depend on the kind and amount of other excipients used, as well as the particular active pharmaceutical ingredient used.

Classes of pharmaceutically acceptable excipients other than the at least one pharmaceutically acceptable sugar include diluents, binders, lubricants, glidants, disintegrants, wetting agents and coloring and flavoring agents. Common diluents are microcrystalline cellulose (e.g. Avicel®), lactose and starch among many others well known in the art. Binders also may be included in tablet formulations to help hold the tablet together after compression. Some typical binders are carboxymethylcellulose sodium, ethylcellulose, gelatin, hydroxypropyl cellulose (e.g. Klucel®), hydroxypropyl methyl cellulose (e.g. Methocel®), povidone (e.g. Kollidon®, Plasdone®), sodium alginate and starch among many others well known in the art. A tablet may further include a disintegrant to accelerate disintegration of the tablet in the patient's stomach. Disintegrants may typically include croscarmellose sodium, crospovidone (e.g. Kollidon®, Polyplasdone®), microcrystalline cellulose, pregelatinized starch, sodium starch glycolate (e.g. Explotab®) among many others well known in the art. A pharmaceutical composition for making compressed tablets may further include glidants, lubricants, flavorings, colorants and other commonly used pharmaceutically acceptable excipients.

Within the scope of the present invention is a novel process of preparing a pharmaceutical granulate that includes the steps of combining an active pharmaceutical ingredient having poor water solubility with a solution, preferably aqueous, of at least one pharmaceutically acceptable sugar and optionally at least one or, preferably more than one, pharmaceutically acceptable excipients other than the at least one pharmaceutically acceptable sugar; drying the product of the combining step; and comminuting the dried product. Suitable pharmaceutically acceptable excipients include a polymer or copolymer of vinyl pyrrolidone and a wetting agent, for example sodium lauryl sulfate. The combining can be by any mixing or dispersing means as is known in the art. For example, weighed ingredients, including the aqueous solution of the at least one sugar, can be combined using a twin-shell mixer of the Patterson-Kelly type, a planetary mixer of the Glen type, or a high shear/high intensity or high speed mixer of the Henschel, Lodige/Littleford, or Baker-Perkins types, to mention just a few. Use of a high shear/high intensity mixer is the preferred means of combining.

In a preferred embodiment of the process of the present invention, at least one pharmaceutically acceptable excipient that is a polymer of vinyl pyrrolidone is included in the combining step and incorporated into the granulate. Suitable polymers of vinyl pyrrolidone include the povidones and crospovidones, available from, for example, the BASF Corporation of Mt. Olive N.J. or International Specialty Products of Wayne N.J., USA. Povidone is example of preferred polymers of vinyl pyrrolidone. Preferably, microcrystalline cellulose (e.g., Avicel®, available from FMC Corporation)

is included in the combining step and is incorporated in the granulate.

Following the combining step, the product of the combining step can be dried, for example in a tray drier or fluidized bed drier, optionally sieved, then comminuted to obtain the granulate. The comminuting can be by any means known in the art, for example milling. A Fitzpatrick mill with 0.5 mm screen is suitable for use in the comminuting step. Those who routinely use this type of equipment will know to optimize the time and intensity of comminuting such that additional comminuting does not result in a significant further increase in the rate or extent of dissolution of the API incorporated in the granulate. The rate and extent of dissolution are preferably measured on tablets compressed from granulate and using methods well known in the art and published, for example, in the United States Pharmacopeia.

In a preferred embodiment, collodial silicon dioxide is blended with the dried combined product prior to comminution. The blending can be by any means known in the art, for example with a planetary mixer or high speed mixer.

The granulate obtained can be used directly, or it can be blended with one or more additional pharmaceutically acceptable excipients prior to use. Preferably, granulate is blended with lubricant prior to use, for example prior to being compressed into tablets.

One skilled in the art of pharmaceutical formulation will know to optimize the kinds and amounts of the active pharmaceutical ingredient (API) having poor water solubility and pharmaceutically acceptable excipients depending on the dosage form to be made and the combining and tableting or capsule filling equipment available. In a preferred combining step, the following are combined in a high shear mixer:
from about 0.3 to about 75 wt.-% of the API having poor water solubility,
from about 5 to about 45 wt.-% of diluent(s),
from about 5 to about 15 wt.-% of disintegrant(s),
from about 0.5 to about 8 wt.-% binder(s),
from about 1 to about 10 wt.-% of wetting agent(s), and
from about 1 to about 50 wt-% of the solution (ca. 1:1, sugar weight:solvent weight) of the at least one pharmaceutically acceptable sugar.
   In the above preferred combining step, about 10 to about 60 wt.-% of API having poor water solubility is sometimes used. The at least one pharmaceutically acceptable sugar used in the preferred combining step above is preferably lactose. An aqueous solution of lactose is particularly preferred as the solution of sugar used in the above preferred combining step. A person skilled in the art can optimize the amount of the aqueous lactose solution used to obtain a mixture with the desired consistency and comminuting characteristics. The product from combining the above ingredients is dried, blended with a glidant (about 0.5 to 1.5 wt.-% based on the weight of the product from combination), and the resultant mixture comminuted using a suitable mill exemplified by a Fitzpatrick impact mill. The comminuted mixture is processed directly (e.g. pressed into tablet cores), or it can be and preferably is blended with lubricant before processing.

The present invention is illustrated with the following non-limiting examples (e.g. see Table 1). Example 1 and 2 are for comparison purposes. Examples 3 and 4 are working examples.

### EXAMPLE 1

Experimental batches, numbers K-31049 and K-31050, were manufactured using a direct compression method. The dry ingredients were dry mixed in a blender and compressed into tablets. The dissolution rates of the resultant tablets were too low, i.e. only about 50% of the active pharmaceutical ingredients dissolved after 45 min, when tested in 1000 mL of 0.05 M aqueous SLS solution, padddle at 75 rpm, at 37° C.

### EXAMPLE 2

Experimental batches, numbers R-00419 and K-31112, were manufactured by wet granulation. The batches were manufactured using a high shear mixer and fluidized bed drier. The extragranular excipients were added to the milled granulate and mixed in a blender. Tablet cores were compressed. Batch R-00419 was manufactured using purified water as a granulation liquid. The resultant tablet's dissolution rate was too low in that only about 58% of the active pharmaceutical ingredient dissolved after 45 min. Batch K-31112 was manufactured using Alcohol 95% as a granulation liquid. The resultant tablet's dissolution rate was also too low in that only about 55% of the active pharmaceutical ingredient dissolved after 45 min on average when tested in 1000 mL of 0.05 M aqueous SLS solution, padddle at 75 rpm, at 37° C.

### EXAMPLE 3 (WORKING EXAMPLE)

Experimental batch K-31557 was manufactured by using a solution of lactose monohydrate in purified water as a granulation solution. The formulation ingredients (bicalutamide, microcrystalline cellulose, povidone, croscarmellose sodium and sodium lauryl sulfate) were combined in a high speed mixer with a solution (1:1, lactose monohydrate wt:water wt) of lactose monohydrate in purified water. The product from the combining step was dried, blended with colloidal silicon dioxide, and milled in a Fitzpatrick impact mill. The granulate so obtained was blended with microcrystalline cellulose and magnesium stearate and compressed into tablet cores in the usual way and the tablet cores were coated.

TABLE 1

| | Batch No. | | | | |
|---|---|---|---|---|---|
| | K-31049 | K-31050 | R-00419 | K-31112 | K-31557 |
| | Direct | Direct | Wet | Wet | Wet |
| Ingredient | compression | compression | granulation | granulation | granulation |
| Bicalutamide | 50.0 | 50.0 | 50.0 | 50.0 | 50.0 |
| Avicel PH 102 | 20.0 | 20.0 | — | 30.0 | 21.0 |
| (Microcrystalline | | | | | |
| Cellulose NF) | | | | | |
| Aerosil 200 | 3.0 | 3.0 | — | — | 2.0 |
| (Colloidal Silicon | | | | | |
| Dioxide NF) | | | | | |
| Lactose Monohydrate | 30.8 | 30.8 | 59.8 | 24.0 | 35.0 |
| NF 200Mesh | | | | | |
| PVP K-30 | 3.0 | 3.0 | 4.0 | 2.0 | 1.5 |
| Povidone USP | | | | | |
| Ac-Di-Sol | — | — | — | — | 12.5 |
| (Croscarmellose | | | | | |
| Sodium NF) | | | | | |
| Sodium Starch | 20.0 | 20.0 | 13.0 | 20.0 | — |
| Glycolate NF | | | | | |
| Sodium Lauryl Sulfate | — | — | — | 2.0 | 4.0 |
| NF | | | | | |
| Magnesium Stearate | 1.2 | 1.2 | 1.2 | 2.0 | 2.0 |
| NF | | | | | |

The dissolution results are shown in Table 2. the dissolution rates were determined in 1000 mL of 0.05 M aqueous SLS solution, padddle at 75 rpm, at 37° C.

TABLE 2

| | 15 min | 30 min | 45 min |
|---|---|---|---|
| K-31049 | 29% | 40% | 48% |
| K-31050 | 31% | 45% | 53% |
| R-00419 | 39% | 51% | 58% |
| K-31112 | 41% | 50% | 55% |
| K-31557 (working example) | 79% | 95% | 97% |

The dissolution results presented in Table 2 are averages of several tablets, so the dissolution rates of individual tablets might lie above or below the average value.

### EXAMPLE 4 (WORKING EXAMPLE)

Formulation 1

A fenofibrate composition, Formulation 1, was made by the wet granulation process of the present invention. The ingredients in Table 3 were wet granulated and then compressed into tablets each weighing 750 mg.

The process of preparing Formulation1 is an example of applying the basic concept of the invention using a solution of lactose (lactose:water, 1:1, v:v) as a binder in the granulation process, followed by drying the mixture and milling the resulting granules. All other non-API components in the granulation mixture may have an effect on the final results but are not critical for applying the concept of the invention and therefore can be replaced (by different components of the same type) or partially omitted. The weight ratios of the granulation components versus the API can be higher or lower than that in Formulation 1. More preferably, the weight ratios are higher than that in Formulation 1 in order to at least maintain or even increase the dissolution rate compare to the dissolution of Formulation 1.

The amount and concentration of the lactose solution used are important. The making of Formulation 1 used a solution of lactose (lactose: water, 212 mg:212 mg) equal to 424 mg which is 40% of the total granulation mixture (solids and water) by weight. In general, the solution of the at least one pharmaceutically acceptable sugar can be between about 15 to about 60%, by weight, of the total granulation mixture, and more preferably between about 35 to about 50% of the total granulation mixture. An increase in the amount of lactose (added as a solution) used can further improve the final results of the invention. For example, increasing the total amount of the granulation solution from 424 mg to 500 mg (lactose: water, 250 mg:250 mg) while maintaining the amounts of the other ingredients constant may improve the dissolution. On the other hand, reducing the total amount of the granulation solution from 424 mg to 250 mg (lactose:water, 125 mg:125 mg) while maintaining the amounts of the other ingredients constant may reduce the dissolution rate. In Example 4, the granules were intensively milled with Fitzmill™ Communitor equipped with a 0.5 mm screen resulting in powder with particle size distribution shown in Table 4, wherein the particle size was determined with sonic filter methodology using ATM Sonic filter or GilsonAutosiever GA equipped with sieves of 60, 80,100,140,170, and Pan.

TABLE 3

| | Weight | Approx. |
|---|---|---|
| Ingredient | (mg/tablet) | Weight Percent |
| Part I: | | |
| Fenofibrate | 160 | 21.3 |
| Polyvinylpyrrolidone | 60 | 8.0 |
| (PVP K-30) | | |
| Sodium Starch Glycolate | 48 | 6.4 |
| Croscarmellose Sodium | 48 | 6.4 |
| (Ac-di-sol ™) | | |
| Crospovidone | 48 | 6.4 |
| Microcrystaline Cellulose | 139.5 | 18.6 |
| (Avicel) | | |
| Part II: | | |
| Sodium Lauryl | 15 | 2.0 |
| Sulfate (SLS) | | |
| Lactose | 212 | 28.3 |
| Part III: | | |
| Aerosil | 7.5 | 1.0 |
| Part IV: | | |
| Pruv (Sodium Stearyl | 12 | 1.6 |
| Fumarate) | | |

The granulates were prepared with a method comprising the following steps:
1. Part I ingredients were thoroughly blended.
2. Lactose of Part II was dissolved in 212 mg of water heated to about 70° C.
3. SLS of Part II was dissolved in about 10 mg of water.
4. The blend of step 1 was granulated by adding the lactose and SLS solutions of steps 2 and 3 to form granules.
5. The granules of step 4 were dried in a Fluidized Bed Drier (FBD) (inlet air 55° C., outlet air Not More Than 40° C.).
6. Aerosil of Part III was blended with the dried granules of step 5 and then milled with Fitzmill™ fitted with a 0.5 mm aperture screen.
7. The Part IV ingredients were then blended with the milled granules of step 6 for about 2 minutes to form a final blend.

8. The final blend was compressed into tablets.

TABLE 4
Particle Size Distribution

| | Retained on (%) | | | | |
|---|---|---|---|---|---|
| | | | Lot 3 | Lot 4 | Lot 5 |
| Sieve size | Lot 1 | Lot 2 | Lot no | Lot no | Lot no |
| (mesh) | k-29740 | k-29738 | F15001 | F15002 | F15003 |
| 60 | 12 | 9.7 | 9.9 | 10.3 | 10.7 |
| 80 | 15 | 13.8 | 11.3 | 13.3 | 13.3 |
| 100 | 7.0 | 6.9 | 3.4 | 6.8 | 6.7 |
| 140 | 14.5 | 17.9 | 13.7 | 11.8 | 11.1 |
| 170 | 7.1 | 6.8 | 3.5 | 5.2 | 5.1 |
| PAN | 43.3 | 43.9 | 57.1 | 52.7 | 53.3 |
| Total | 98.8 | 99.0 | 98.9 | 100.1 | 100.2 |

Table 4 shows that at least about 72% of the milled granules passed through the 80 mesh screen and at least about 66% passed through the 100 mesh screen.

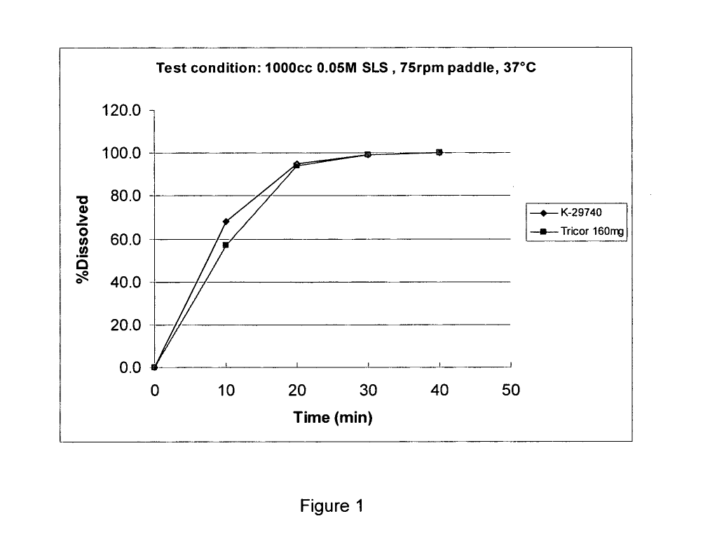

The dissolution profile of the tablets of Lot 1 (K-29740) of Formulation 1 was tested in 1000 mL of 0.05 M aqueous SLS solution, paddle (Apparatus II) at 75 rpm and 37° C. For comparison, the dissolution profile of commercial fenofibrate tablets, Tricor 160 mg, was also tested in the same way. The dissolution profiles obtained are presented in Table 5 and shown graphically in FIG. 1.

TABLE 5
(Dissolution Profile)

| Time (min) | K-29740 | Tricor 160 mg |
|---|---|---|
| 0 | 0.0 | 0.0 |
| 10 | 68.0 | 57.0 |
| 20 | 95.0 | 94.0 |
| 30 | 99.0 | 99.0 |
| 40 | 100.0 | 100.0 |

The pharmaceutical granulates of the present invention comprising the active pharmaceutical ingredient, e.g. fenofibrate, of poor aqueous solubility intimately associated with the at least one pharmaceutically acceptable sugar can have particle size distribution in that at least about 70% passes through a 80 mesh screen, at least about 60% passes through a 100 mesh screen and at least about 50% passes through a 140 mesh screen.

The solid pharmaceutical formulations, e.g., tablets, of the present invention can display dissolution properties such that after about 10 minutes at least about 50%, preferably at least about 60%, is dissolved; after about 20 minutes at least about 70%, more preferably at least about 75%, is dissolvoed; after about 30 minutes at least about 80%, more preferably at least about 85%, is dissolved; and after about 40 minutes at least about 90%, preferably at least about 95%, and more preferably about 98% to about 100%, is dissolved, when determined under conditions at least as stringent as 1000 mL of 0.05 M aqueous SLS solution, padddle at 75 rpm, at 37° C.

What is claimed is:

1. A granulate for pharmaceutical composition, comprising an active pharmaceutical ingredient having a poor water solubility intimately associated with at least one pharmaceutically acceptable sugar, wherein the active pharmaceutical ingredient has a solubility in water of less than about 20 mg/ml, with the proviso that when the active pharmaceutical ingredient is fenofibrate the at least one pharmaceutically acceptable sugar is not lactose.

2. The granulate of claim 1, further comprising at least one pharmaceutically acceptable excipient other than the at least one pharmaceutically acceptable sugar.

3. The granulate of claim 2, wherein the at least one pharmaceutically acceptable excipient other than the at least one pharmaceutically acceptable sugar is selected from the group consisting of diluents, binders, disintegrants, wetting agents, lubricants, glidants, coloring agents and flavoring agents.

4. The granulate of claim 3, wherein the diluents are microcrystalline cellulose, lactose and starch, the binders are carboxymethylcellulose sodium, ethylcellulose, gelatin, hydroxypropyl cellulose, hydroxypropyl methyl cellulose, povidone, sodium alginate and starch, and the disintegrants are croscarmellose sodium, crospovidone, microcrystalline cellulose, pregelatinized starch and sodium starch glycolate.

5. The granulate of claim 3, wherein the at least one pharmaceutically acceptable excipient other than the at least one pharmaceutically acceptable sugar is selected from the group consisting of polymers or copolymers of vinyl pyrrolidone, croscarmellose sodium, microcrystalline cellulose and sodium lauryl sulfate.

6. The granulate of claim 5, wherein the at least one pharmaceutically acceptable excipient other than the polymers or copolymers of vinyl pyrrolidone are selected from povidone and crospovidone.

7. The granulate of claim 6, wherein the at least one pharmaceutically acceptable excipient other than the at least one pharmaceutically acceptable sugar comprises povidone, microcrystalline cellulose and sodium lauryl sulfate.

8. The granulate of claim 7, wherein the at least one pharmaceutically acceptable excipient other than the at least one pharmaceutically acceptable sugar comprises povidone, microcrystalline cellulose, sodium lauryl sulfate, croscarmellose sodium and colloidal silicon dioxide.

9. The granulate of claim 1, wherein the at least one pharmaceutically acceptable sugar is selected from mannitol, sorbitol, glucose, fructose, galactose, sucrose, maltose, isomaltose, cellobiose, melibiose, gentiobiose and lactose.

10. The granulate of claim 1, wherein the at least one pharmaceutically acceptable sugar comprises a pharmaceutically acceptable disaccharide.

11. The granulate of claim 10, wherein the at least one pharmaceutically acceptable sugar comprises a pyranosyl pyranose.

12. The granulate of claim 11, wherein the pyranosyl pyranose is selected from maltose, isomaltose, cellobiose, lactose, melibiose and gentiobiose.

13. The granulate of claim 12, wherein the at least one pharmaceutically acceptable sugar is lactose.

14. The granulate of claim 1, wherein the active pharmaceutical ingredient is selected from fenofibrate, bicalutamide, atorvastatin, fluvastatin, simvastatin, candesartan, ezetimibe, oxcarbazepine, meloxicam, celecoxib, rofecoxib, valdecoxib, raloxifene, aripiprazole and glyburide.

15. The granulate of claim 14, wherein the active pharmaceutical ingredient is bicalutamide.

16. The granulate of claim 15, wherein the at least one pharmaceutically acceptable excipient other than the at least one pharmaceutically acceptable sugar comprises povidone, microcrystalline cellulose, sodium lauryl sulfate, croscarmellose sodium and colloidal silicon dioxide.

17. The granulate of claim 16, wherein the at least one pharmaceutically acceptable sugar is lactose or lactose monohydrate, and wherein the at least one pharmaceutically acceptable excipient other than the at least one pharmaceutically acceptable sugar comprises povidone, microcrystalline cellulose, sodium lauryl sulfate, croscarmellose sodium, colloidal silicon dioxide and magnesium stearate.

18. A pharmaceutical granulate produced by a process comprising
(a) combining
an active pharmaceutical ingredient having poor water solubility,
a solution comprising at least one pharmaceutically acceptable sugar and,
optionally, at least one pharmaceutically acceptable excipient other than the at
least one pharmaceutically acceptable sugar
to form a mixture, wherein the active pharmaceutical ingredient has a water solubility of less than about 20 mg per ml of water, and wherein the solution comprises the at least one pharmaceutically acceptable sugar and at least one solvent;
(b) drying the mixture; and
(c) comminuting the product of step (b) to obtain the pharmaceutical granulate.

19. The pharmaceutical granulate of claim 18, wherein
the at least one solvent is water; and
the at least one pharmaceutically acceptable sugar is lactose.

20. The pharmaceutical granulate of claim 19, wherein the aqueous solution of the at least one pharmaceutically acceptable sugar mixed with the active pharmaceutical ingredient in step (a) contains about 1:1, weight of the at least one pharmaceutically acceptable sugar:weight of water.

21. The pharmaceutical granulate of claim 18, wherein the following substances are combined in step (a):
from about 0.3 to about 75 wt.-% of the active pharmaceutical ingredient,
from about 5 to about 45 wt.-% of a diluent,
from about 5 to about 15 wt.-% of a disintegrant,
from about 0.5 to about 8 wt.-% of a binder,
from about 1 to about 10 wt.-% of of a wetting agent, and
from about 1 to about 50 wt-% of the solution (sugar+solvent) of the at least one pharmaceutically acceptable sugar (about 1:1, sugar weight:solvent weight).

22. The granulate of claim 1, wherein the weight ratio of the at least one pharmaceutically acceptable sugar and the active pharmaceutical ingredient having poor water solubility in the granulate is about 0.1:1 to about 1000:1.

23. The granulate of claim 22, wherein the weight ratio of the at least one pharmaceutically acceptable sugar and the active pharmaceutical ingredient having poor water solubility in the granulate is about 0.1:1 to about 100:1.

24. The granulate of claim 23, wherein the weight ratio of the at least one pharmaceutically acceptable sugar and the active pharmaceutical ingredient having poor water solubility in the granulate is about 0.1:1 to about 10:1.

25. The granulate of claim 24, wherein the active pharmaceutical ingredient having poor water solubility is bicalutamide.

26. The granulate of claim 14, wherein the active pharmaceutical ingredient is fenofibrate.

27. A granulate for pharmaceutical composition, comprising an active pharmaceutical ingredient having a poor water solubility comprised of fenofibrate intimately associated with at least one pharmaceutically acceptable sugar, and the particle size distribution of the granulate is such that at least about 75 percent of the granulate pass through an 80 mesh screen.

28. The granulate of claim 27 where at least about 66 percent of the granulate passes through a 100 mesh screen.
